(19) United States
(12) Patent Application Publication
(10) Pub. No.: US 2024/0189480 A1
(43) Pub. Date: Jun. 13, 2024

(54) POLYPHENOL COATING AND PREPARATION METHOD AND USE THEREOF

(71) Applicant: Anhui Medical University, Hefei City (CN)

(72) Inventors: Hua QIU, Hefei City (CN); Zhaojia LIANG, Hefei City (CN); Quanli LI, Hefei City (CN); Zirui CHEN, Hefei City (CN); Jialong CHEN, Hefei City (CN); Xiangyang LI, Hefei City (CN); Shuang TAN, Hefei City (CN)

(21) Appl. No.: 18/295,378

(22) Filed: Apr. 4, 2023

(30) Foreign Application Priority Data
Dec. 12, 2022 (CN) 202211623791.4

(51) Int. Cl. A61L 27/36

(52) U.S. Cl. CPC A61L 27/3637

(57) ABSTRACT

The present disclosure relates to the technical field of coatings. In order to prepare a polyphenol coating more efficiently and gently on the surface of a material, the present disclosure provides a preparation method of a polyphenol coating, including the following steps: (1) preparing a Tanfloc aqueous solution; (2) adjusting the Tanfloc aqueous solution obtained in step (1) to be in a colloidal suspension state to obtain a modified solution; and (3) placing a carrier material in the modified solution prepared in step (2) to obtain a modified coating on a surface of the carrier material. In the present disclosure, by the preparation method of a polyphenol coating, the polyphenol coating can be efficiently prepared on a material surface under mild conditions.

### CROSS REFERENCE TO RELATED APPLICATION

This patent application claims the benefit and priority of Chinese Patent Application No. 202211623791.4, filed with the China National Intellectual Property Administration on Dec. 12, 2022, the disclosure of which is incorporated by reference herein in its entirety as part of the present application.

### TECHNICAL FIELD

The present disclosure relates to the technical field of coatings, in particular to a polyphenol coating and a preparation method and use thereof.

### BACKGROUND

In the field of medical technology, bioactive coatings have been widely used in the surface modification of medical devices, such as the organic or ceramic coatings on bone/dental implants. Bioactive coating on the surface of medical implants can increase biocompatibility of the implant surface, and also allow the implant surface to have certain functions, such as antibacterial properties, regulation of inflammatory response and cell behavior.

However, for the prior art, most bioactive coating techniques rely on a long preparation time, complex preparation steps or harsh preparation conditions. A few reported methods of rapidly preparing coatings under mild conditions either have insufficient coating thickness, or need to introduce other molecules or ions to conduct co-deposition to improve coating efficiency. In addition, many rapid coating preparation strategies place high demands on the cleanliness of the substrate surface. Once the substrate surface is contaminated, coating deposition can be problematic. In some cases, infection and inflammation occur after implantation occurs in the implant, and surgeons need to remove contaminants on the implant's surface to restore its affinity with surrounding tissues. However, contaminants on the implant surface cannot be completely removed when cleaning in vivo, which leads to the challenge of quick building a bioactive coating on the implant in situ.

Tanfloc, as a polymeric polyphenolic substance, is widely used in sewage treatment, showing desirable biocompatibility and antioxidant properties. At present, some scholars conduct co-deposition on the Tanfloc with a variety of functional molecules (such as heparin and hyaluronic acid) on the surface of implants or glass to obtain coatings, and have achieved anticoagulation effects and growth promotion of osteoblasts. However, the Tanfloc coating process reported so far requires layer-by-layer self-assembly or with other (potentially toxic) molecules. These methods are cumbersome and have a total preparation time of at least 40 minutes, limiting its clinical application that requires a short surgical time window (e.g. Surface cleaning of infected implants is usually done within minutes, and if it is too long, it can cause patient discomfort and increase the risk of surgical failure). Besides, in-situ construction of coatings on implant surfaces in vivo requires the body to withstand the conditions under which the coating is prepared. However, the reported Tanfloc coating needs acidic environment at a pH value of 5 to 5.5 and a hypotonic environment that may cause significant tissue irritation or the patient's discomfort. As a results, the prior art cannot realize the simple and rapid preparation of the coating under mild conditions.

### SUMMARY

An objective of the present disclosure is to provide a polyphenol coating and a preparation method thereof, such that the polyphenol coating can be rapidly prepared on a clean or contaminated material surface under mild conditions.

The present disclosure provides a preparation method of a polyphenol coating, including the following steps:
(1) preparing a Tanfloc aqueous solution with 0.05 mg/ml to 20 mg/ml of Tanfloc by concentration;
(2) adjusting the Tanfloc aqueous solution obtained in step (1) to be in a colloidal suspension state to obtain a modified solution; and
(3) placing a carrier material in the modified solution prepared in step (2), and conducting incubation in an ultrasonic or vibrating environment for greater than or equal to 1 min to obtain a modified coating on a surface of the carrier material. The carrier material includes, but is not limited to, common medical materials such as metals and polymers.

Further, in step (2), the Tanfloc aqueous solution is adjusted with an alkaline solution, such that the modified solution is at a pH value of 5.5 to 8.5. The alkaline solution includes, but is not limited to, sodium hydroxide, potassium hydroxide, and ammonium hydroxide.

Further, in step (2), the Tanfloc aqueous solution is treated with a salt solution, such that the Tanfloc aqueous solution forms a colloid. The salt solution includes, but is not limited to, sodium chloride and potassium chloride. The salt solution can make the Tanfloc aqueous solution form a colloid, such that there is no specific limitation on a concentration and a dosage of the salt solution and the salt solution can be added into the Tanfloc aqueous solution to form the colloid.

The technical solution of the examples in the present disclosure has at least the following advantages and beneficial effects: in the polyphenol coating and the preparation method thereof:

(1) In terms of preparation conditions, the preparation method replaces a preparation environment from acidic to neutral, without introducing other molecules, which is more conducive to in vivo preparation; meanwhile, the coating is prepared by colloidal suspension, which avoids the disadvantages of tedious LbL coating process reported in the past, without introducing other molecules, which realizes rapid preparation of coating under mild conditions.

(2) In terms of technical sensitivity of the preparation, the preparation method adjusts the pH value to change the Tanfloc from a solution state into suspended small colloidal particles, which achieves a better adsorption effect and reduces the technical sensitivity, such that a coating can be formed on the surface even in the presence of contamination on the implant.

(3) In terms of thickness and loading capacity of the prepared coating, compared with traditional polyphenol coatings (monomolecular coatings of 1 nm to 2 nm), the preparation method adopts polyphenol after colloidal suspension to obtain a thicker macromolecular polyphenol coating (approximately 10 nm per layer) with greater loading capacity.

### BRIEF DESCRIPTION OF THE DRAWINGS

FIG. 1 shows a structural formula of Tanfloc used in Examples 1 to 6 of the present disclosure;

FIG. 2 shows an X-ray photoelectron spectroscopy (XPS) result provided by Example 1 of the present disclosure;

FIG. 3 shows a contact angle result provided by Example 1 of the present disclosure;

FIG. 4 shows a Zeta potential result provided by Example 1 of the present disclosure; and

FIG. 5A-B show an XPS result provided by Example 4 of the present disclosure.

### DETAILED DESCRIPTION OF THE EMBODIMENTS

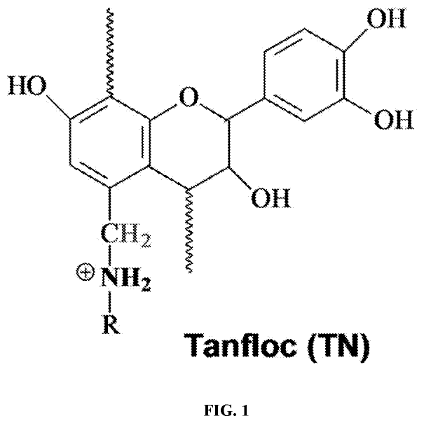

FIG. 1 shows a structural formula of Tanfloc used in Examples 1 to 6.

### Example 1

In this example, a preparation method of a polyphenol coating included the following steps:

(1) Cleaning of a carrier material: a polished carrier material was placed into an ultrasonic cleaner, and washed in acetone and absolute alcohol separately for 5 min to 10 min by an ultrasonic wave at 15 kHz to 30 kHz, and then dried.

(2) A 1 mg/ml Tanfloc solution was prepared, adjusted to a pH value of 7.0 with 1 M sodium hydroxide, and the solution was immersed into a colloid to be in a colloidal suspension state to form a modified solution; and a cleaned carrier material was immersed in the modified solution for 10 min under an ultrasonic environment.

(3) The un-adsorbed Tanfloc was removed by washing with RO water to obtain a modified coating.

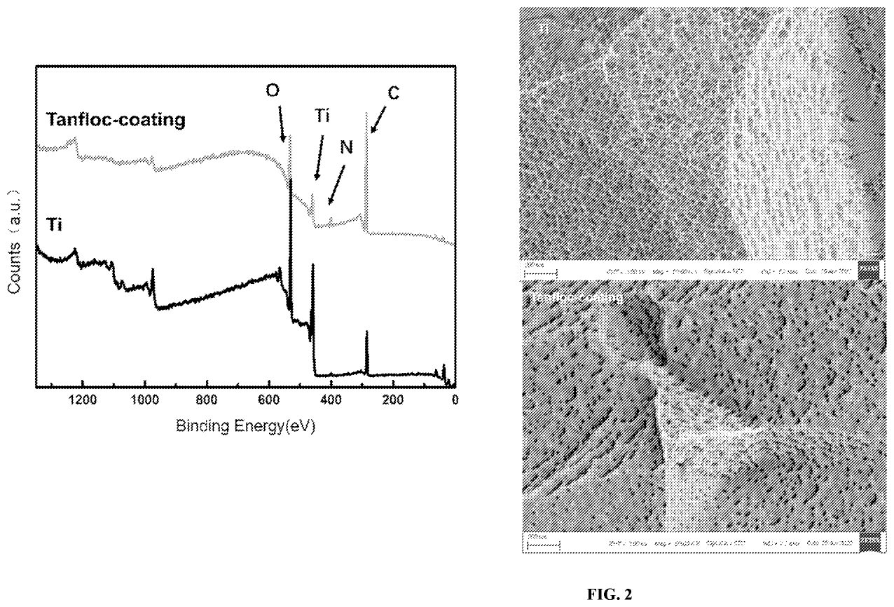

A coating state on the surface of the carrier material was detected by XPS, and the results were shown in FIG. 2. It was seen from FIG. 2 that the XPS results on a left showed that a Ti/C peak was inverted, and a new N element peak appeared, proving that Tanfloc in a colloidal suspension state could form a coating on the surface of medical implants; and SEM results on a right side showed that the formed coating had a thickness close to 10 nm. It was confirmed that the preparation method of the present disclosure could quickly form a coating on the surface of a medical implant under mild conditions.

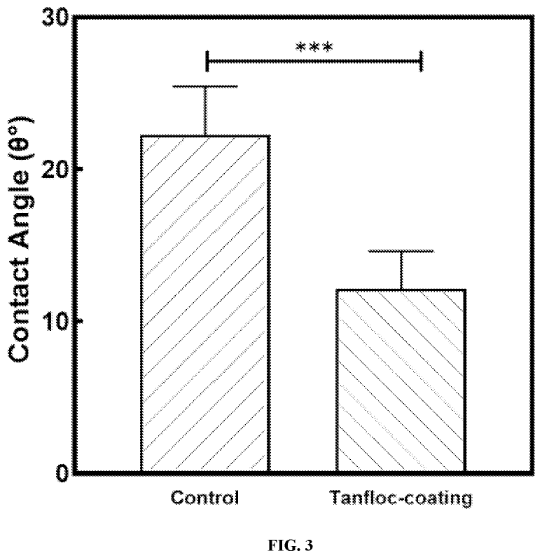

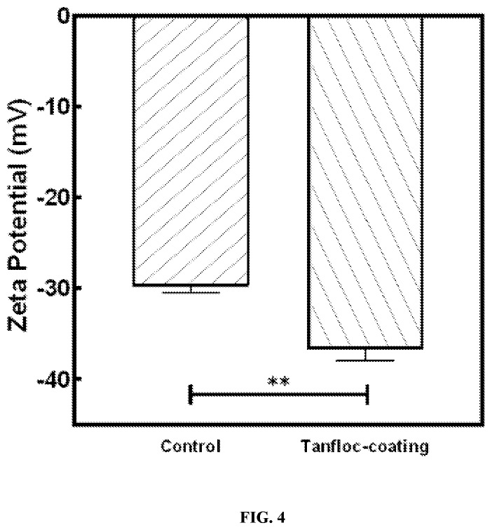

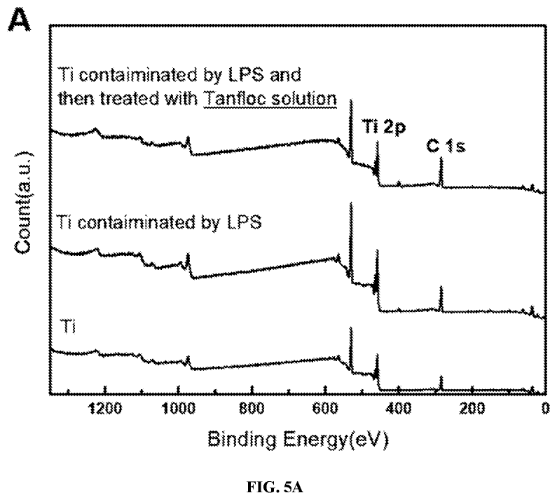

FIG. 3 showed changes of a contact angle before and after the polyphenol coating was deposited on the carrier material. It was seen from the results that the contact angle on the surface of the polyphenol coating-coated carrier material was significantly reduced. FIG. 4 showed surface Zeta potential changes before and after the polyphenol coating was deposited on the carrier material. It was seen from the results that the Zeta potential of the polyphenol coating surface was significantly reduced. The above results indicated that the polyphenol coating not only formed a coating on the surface of the carrier material, but also significantly changed the physical and chemical properties of the material surface.

### Example 2

In this example, a preparation method of a polyphenol-copper ion coating included the following steps:

(1) Cleaning of a carrier material: a polished carrier was placed into an ultrasonic cleaner, and washed in acetone and absolute alcohol separately for 5 min to 10 min by an ultrasonic wave at 15 kHz to 30 kHz, and then dried.

(2) A 1 mg/ml Tanfloc solution was prepared, adjusted to a pH value of 7.0 with 6 M sodium hydroxide, and the solution was immersed into a colloid to be in a colloidal suspension state to form a modified solution; and a cleaned carrier material was immersed in the modified solution for 10 min under an ultrasonic environment.

(3) The un-adsorbed Tanfloc was removed by washing with RO water to obtain a modified coating.

(4) The carrier material containing the modified coating was immersed in a 1 mg/ml cupric chloride solution for 3 min.

(5) The un-adsorbed ions were removed by washing with RO water to obtain a Tanfloc-copper ion composite coating.

### Example 3

In this example, a preparation method of a polyphenol coating included the following steps:

(1) Cleaning of a carrier material: a polished carrier was placed into an ultrasonic cleaner, and washed in acetone and absolute alcohol separately for 5 min to 10 min by an ultrasonic wave at 15 kHz to 30 kHz, and then dried.

(2) A 1 mg/ml Tanfloc solution was prepared, added with an equal amount of normal saline, and the solution was immersed into a colloid to be in a colloidal suspension state to form a modified solution; and a cleaned carrier material was immersed in the colloidal suspension for 10 min under an ultrasonic environment.

(3) The un-adsorbed Tanfloc was removed by washing with RO water to obtain a coating.

### Example 4

In this example, a preparation method of a polyphenol coating on a contaminated implant surface included the following steps:

(1) Cleaning of a carrier material: a polished carrier was placed into an ultrasonic cleaner, and washed in acetone and absolute alcohol separately for 5 min to 10 min by an ultrasonic wave at 15 kHz to 30 kHz, and then dried.

(2) Preparation of a contaminated carrier material surface: the carrier material was immersed in a 5 μg/ml LPS aqueous solution for 1 h, the un-adsorbed LPS was removed by rinsing with RO water, and the carrier material was dried to obtain a carrier material contaminated by LPS.

(3) A 1 mg/ml Tanfloc solution was prepared, adjusted to a pH value of 7.0 with 1 M sodium hydroxide, and the solution was immersed into a colloid to be in a colloidal suspension state to form a modified solution; and the contaminated carrier material obtained in step (2) was immersed into the modified solution under a shaker for 10 min.

(3) The un-adsorbed Tanfloc was removed by washing with RO water to obtain a polyphenol coating.

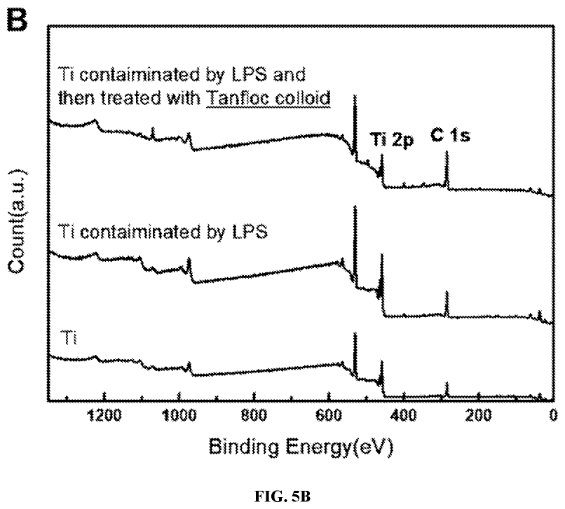

Meanwhile, XPS was conducted to detect the polyphenol coating obtained in this example and the polyphenol coating in a control group (the control group was to directly immerse the carrier material in the Tanfloc solution (non-modified solution)), and the results were shown in FIG. 5A-B. It was seen from FIG. 5A-B that unmodified Tanfloc had less deposition on the surface of medical implants contaminated by LPS (in FIG. 5A, the Ti/C peak failed to reverse); however, the Tanfloc under the modified solution could be deposited in large quantities on the surface of medical implants contaminated by LPS (in FIG. 5B, the Ti/C peak was reversed). It was proved that the preparation method of the present disclosure could form a polyphenol coating on the surface of medical implants even after being contaminated by LPS (a common substance from bacterial contamination).

### Example 5

In this example, a preparation method of a polyphenol coating on an implant in vivo included the following steps:

(1) A 1 mg/ml Tanfloc solution was prepared, adjusted to a pH value of 7.0 with 1 M sodium hydroxide, and the solution was immersed into a colloid to be in a colloidal suspension state to form a modified solution;

(2) after exposing an implant in vivo during operation, a surface of the implant was rinsed for 3 min to 5 min with the modified solution in step (1), while excess liquid was removed using a suction device; and

(3) The excess liquid was removed by rinsing with normal saline.

### Example 6

In this example, a preparation method of a polyphenol coating on a contaminated intraoral implant surface included the following steps:

(1) A 1 mg/ml Tanfloc solution was prepared, adjusted to a pH value of 7.0 with 1 M sodium hydroxide, and the solution was immersed into a colloid to be in a colloidal suspension state to form a modified solution;

(2) after exposing threads of the intraoral implant during operation, a surface of the intraoral implant was rinsed with the modified solution for 3 min to 5 min using an ultrasonic irrigator, while excess liquid was removed using a suction device; and

(3) The excess liquid was removed by rinsing with normal saline.

The above examples are merely preferred examples of the present disclosure and are not intended to limit the present disclosure, and various changes and modifications may be made to the present disclosure by those skilled in the art. Any modifications, equivalent substitutions, improvements, etc. made within the spirit and scope of the present disclosure should be included within the protection scope of the present disclosure.

What is claimed is:

1. A preparation method of a polyphenol coating, comprising the following steps:
(1) preparing a Tanfloc aqueous solution;
(2) adjusting the Tanfloc aqueous solution obtained in step (1) to be in a colloidal suspension state to obtain a modified solution; and
(3) placing a carrier material in the modified solution prepared in step (2) to obtain a modified coating on a surface of the carrier material.

2. The preparation method of a polyphenol coating according to claim 1, wherein in step (2), the Tanfloc aqueous solution is adjusted with an alkaline solution, such that the modified solution is at a pH value of 5.5 to 8.5.

3. The preparation method of a polyphenol coating according to claim 1, wherein in step (2), the Tanfloc aqueous solution is treated with a salt solution, such that the Tanfloc aqueous solution forms a colloid.

4. The preparation method of a polyphenol coating according to claim 1, wherein in step (3), the carrier material is immersed in the modified solution for greater than or equal to 1 min.

5. The preparation method of a polyphenol coating according to claim 1, wherein in step (3), when the carrier material is placed in the modified solution, the modified solution is in an ultrasonic or vibrating environment.

6. The preparation method of a polyphenol coating according to claim 1, wherein in step (1), the Tanfloc aqueous solution has 0.05 mg/ml to 20 mg/ml of Tanfloc.

7. A polyphenol coating prepared by the preparation method of a polyphenol coating according to claim 1.

8. The polyphenol coating according to claim 7, wherein in step (2), the Tanfloc aqueous solution is adjusted with an alkaline solution, such that the modified solution is at a pH value of 5.5 to 8.5.

9. The polyphenol coating according to claim 7, wherein in step (2), the Tanfloc aqueous solution is treated with a salt solution, such that the Tanfloc aqueous solution forms a colloid.

10. The polyphenol coating according to claim 7, wherein in step (3), the carrier material is immersed in the modified solution for greater than or equal to 1 min.

11. The polyphenol coating according to claim 7, wherein in step (3), when the carrier material is placed in the modified solution, the modified solution is in an ultrasonic or vibrating environment.

12. The polyphenol coating according to claim 7, wherein in step (1), the Tanfloc aqueous solution has 0.05 mg/ml to 20 mg/ml of Tanfloc.

13. A method for surface modification of a medical material using the preparation method of a polyphenol coating according to claim 1.

14. The method according to claim 13, wherein in step (2), the Tanfloc aqueous solution is adjusted with an alkaline solution, such that the modified solution is at a pH value of 5.5 to 8.5.

15. The method according to claim 13, wherein in step (2), the Tanfloc aqueous solution is treated with a salt solution, such that the Tanfloc aqueous solution forms a colloid.

16. The method according to claim 13, wherein in step (3), the carrier material is immersed in the modified solution for greater than or equal to 1 min.

17. The method according to claim 13, wherein in step (3), when the carrier material is placed in the modified solution, the modified solution is in an ultrasonic or vibrating environment.

18. The method according to claim 13, wherein in step (1), the Tanfloc aqueous solution has 0.05 mg/ml to 20 mg/ml of Tanfloc.
